(19) United States
(12) Patent Application Publication
(10) Pub. No.: US 2022/0288000 A1
(43) Pub. Date: Sep. 15, 2022

(54) CHEWABLE FORMULATIONS

(71) Applicant: HOFFMAN TECHNOLOGIES LLC, New York, NY (US)

(72) Inventors: Steven Hoffman, Henderson, NV (US); John Rothman, Lebanon, NJ (US)

(73) Assignee: HOFFMAN TECHNOLOGIES LLC, New York, NY (US)

(21) Appl. No.: 17/690,663

(22) Filed: Mar. 9, 2022

Related U.S. Application Data
(60) Provisional application No. 63/158,629, filed on Mar. 9, 2021.

(51) Int. Cl. A61K 31/198; A61K 9/00; A61K 38/095; A61K 31/197; A61K 31/27; A61K 31/4178; A61K 31/135; A61K 31/138; A61K 31/4525; A61K 31/137; A61K 47/26; A61K 47/46; A61K 47/12; A61K 47/02; A61K 47/10; A61K 47/38

(52) U.S. Cl. CPC A61K 31/198; A61K 9/0056; A61K 38/095; A61K 31/197; A61K 31/27; A61K 31/4178; A61K 31/135; A61K 31/138; A61K 31/4525; A61K 31/137; A61K 47/26; A61K 47/46; A61K 47/12; A61K 47/02; A61K 47/10; A61K 47/38

(57) ABSTRACT

The invention relates to tyrosine hydroxylase inhibitor compositions and methods of preparing and administering thereof. Specifically, the invention relates to an oral chewable formulation of a tyrosine hydroxylase inhibitor, particularly α-methyl-DL-tyrosine.

### CROSS-REFERENCE TO RELATED APPLICATION

This application claims the benefit of U.S. Provisional Application No. 63/158,629, filed Mar. 9, 2021, the entirety of which is incorporated by reference herein.

### FIELD OF THE INVENTION

The invention relates to tyrosine hydroxylase inhibitor compositions and methods thereof. Specifically, the invention relates to an oral chewable formulation of a tyrosine hydroxylase inhibitor, particularly α-methyl-DL-tyrosine.

### BACKGROUND OF THE INVENTION

Chewable pharmaceutical compositions comprising at least one pharmaceutically active ingredient, such as chewable tablet formulations or soft chew formulations, are required to be broken and chewed, i.e., mechanically disintegrated, in the mouth of the subject ingesting the composition. Chewable pharmaceutical compositions offer a convenient substitute for conventional (and especially large) oral dosage forms, such as pills/tablets, especially for people having difficulty swallowing (dysphagia) in all age groups, especially the elderly and children. Advantageously, chewable pharmaceutical compositions, such as chewable tablets or gummy compositions, do not require water or liquid to be taken concurrently, which makes chewable tablets more user friendly than conventional tablets and increases patient acceptance through a pleasant taste and improves patient compliance to a recommended course of treatment. Another benefit of chewable pharmaceutical compositions is improved bioavailability of the active pharmaceutical ingredient (thus reducing lag time upon ingestion) by its being released in the mouth upon chewing rather than the disintegration required before absorption in the stomach of a conventional tablet. Chewable pharmaceutical compositions also reduce the risk of drug-induced esophagitis, which occurs when a conventional tablet, i.e., a tablet not formulated for chewing and not intended to be chewed, is lodged in the esophagus and dissolves while in contact with the esophageal lining. The non-drug components of a chewable pharmaceutical composition, i.e., the non-active pharmaceutical ingredients (API), are called excipients.

The main factors in formulating chewable pharmaceutical compositions, including but not limited to chewable tablets, are flow, lubrication, disintegration, organoleptic properties, compressibility, compatibility and stability, of which the organoleptic properties of the active drug, and the chewable tablet “as a whole”, are major issues. Organoleptic is defined herein as an aspect of a substance that a person experiences with the senses of taste, sight (color), odor, and touch (feel). In certain embodiments, the substance may be a chewable oral dosage formulation, including but not limited to chewable tablet, chewable wafer, soft chewable composition, such as a gummy composition (also called a “gummie”), a soft-chew composition or chewing gum, regardless of its geometric form, provided herein.

Tyrosine hydroxylase or tyrosine 3-monooxygenase is the enzyme responsible for catalyzing the conversion of the amino acid L-tyrosine to L-3,4-dihydroxyphenylalanine (L-DOPA). It does so using molecular oxygen (O₂), as well as iron (Fe²⁺) and tetrahydrobiopterin as cofactors.

Tyrosine hydroxylase inhibition can lead to a depletion of dopamine and norepinepherine in the brain due to the lack of the precursor L-Dopa (L-3,4-dyhydroxyphenylalanine) which is synthesized by tyrosine hydroxylase.

Various tyrosine hydroxylase inhibitors, for example, α-methyl-L-tyrosine (metirosine) and α-methyl-DL-tyrosine, are well known in the art

α-methyl-DL-tyrosine is a racemate molecule of α-methyl-L-tyrosine (also known as metyrosine; “metyrosine”), which is an FDA approved drug and currently being sold as DEMSER®.

Although tyrosine hydroxylase inhibitors are commercially available, those skilled in the art have not developed any chewable formulation. To date, no chewable pharmaceutical compositions exist for any of the tyrosine hydroxylase inhibitors, including α-methyl-DL-tyrosine.

Accordingly, there exists a need for chewable formulations of tyrosine hydroxylase inhibitors.

### SUMMARY OF THE INVENTION

In one aspect, the invention provides a chewable pharmaceutical formulation comprising a therapeutically effective amount of a tyrosine hydroxylase inhibitor and a chewability enhancing excipient. In an embodiment, the chewability enhancing excipient may comprise a disintegrant, a taste masking agent or a combination thereof. In some embodiments, the disintegrant may comprise starch and starch derivatives, such as sodium starch glycolate (commercially available as EXPLOTAB®, VIVASTAR® and PRIMOGEL®), cellulose (microcrystalline cellulase (“MCC”), commercially available as AVICEL® PH101 and AVICEL® PH102) and sodium carboxymethyl cellulose (Na-CMC) (and a combination thereof commercially available as RC 591 from SANCEL®) and cellulose derivatives, such as croscarmellose sodium (a crosslinked Na-CMC, commercially available as Ac-Di-Sol®), crosslinked polymers, such as crosslinked polyvinylpyrrolidone (PVP) [also called crospovidone], clays, such as bentonite, alginates, and a cation exchange resin. In various embodiments, the provided chewable tablets formulations comprising AMPT comprise a disintegrant, such as fructose, povidone (polyvinylpyrrolidone (PVP)), a surfactant or combinations thereof. In some embodiments, the disintegrant may comprise a natural gum (also called biopolymer), such as xanthan gum, alginate, chitosan, carrageenan, gellan gum, guar gum, gelatin, agar, alginate, carrageenan(s), such as iota carrageenan and kappa carrageenan, cellulose, gellan gum, gum Arabic, konjac gum, locust bean gum, modified starch, pectin and/or combinations thereof. In various embodiments, the taste masking agent is a flavoring agent, a sweetener, a lipid, an acid or a combination thereof.

In another aspect, the invention provides a method for manufacturing the chewable pharmaceutical formulation comprising a therapeutically effective amount of a tyrosine hydroxylase inhibitor, the method comprising admixing the tyrosine hydroxylase inhibitor and the chewability enhancing excipient; and configuring the mixture into a unit dosage form.

In a further aspect, the invention provides a method for treating a disease or disorder in a subject in need thereof, the method comprising administering a chewable pharmaceutical formulation comprising a therapeutically effective amount of a tyrosine hydroxylase inhibitor to the subject.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment incudes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

The term “stereoisomers” refers to compounds that have identical chemical constitution, but differ as regards the arrangement of the atoms or groups in space. The term “enantiomers” refers to stereoisomers that are mirror images of each other that are non-superimposable.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

The term “inhibitor” as used herein includes compounds that inhibit the expression or activity of a protein, polypeptide or enzyme and does not necessarily mean complete inhibition of expression and/or activity. Rather, the inhibition includes inhibition of the expression and/or activity of a protein, polypeptide or enzyme to an extent, and for a time, sufficient to produce the desired effect.

While not intending to be bound by any particular mechanism of operation, it is believed that the tyrosine hydroxylase inhibitors according to the present invention function by decreasing the amount of adrenaline secreted into the bloodstream.

### Tyrosine Hydroxylase Inhibitor

The tyrosine hydroxylase inhibitor is well known in the art and fully described in, for example, U.S. Patent Application Publications US 2015/0290279, US 2015/0216827, US 2015/0111937, US 2015/0111878, US 2013/0184214, and US 20130183263; U.S. Pat. Nos. 8,481,498, 9,308,188, and 9,326,962; and PCT Patent Application Publication WO2015061328, which are incorporated by reference herein in their entirety. Any suitable tyrosine hydroxylase inhibitor, known to one of skilled in the art, can be used.

In certain embodiments, the tyrosine hydroxylase inhibitor is a tyrosine derivative. The tyrosine derivative can be capable of existing in different isomeric forms, including stereoisomers and enantiomers. The tyrosine derivative can, for example, exist in both L-form or D-form. The tyrosine derivative can, for example, also exist in a racemic form.

Representative tyrosine derivatives include, for example, one or more of methyl (2R)-2-amino-3-(2-chloro-4 hydroxyphenyl) propanoate, D-tyrosine ethyl ester hydrochloride, methyl (2R)-2-amino-3-(2,6-dichloro-3,4-dimethoxyphenyl) propanoate H-D-tyrosine(tBu)-allyl ester hydrochloride, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6-fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6-dichloro-3-hydroxy-4-methoxyphenyl) propanoate, methyl (2R)-2-amino-3-(3-chloro-4-hydroxyphenyl) propanoate, H-DL-tyrosine methyl ester hydrochloride, H-3,5-diiodo-tyrosine methyl ester hydrochloride, H-D-3,5-diiodo-tyrosine methyl ester hydrochloride, H-D-tyrosine methyl ester hydrochloride, D-tyrosine methyl ester hydrochloride, D-tyrosine-methyl ester hydrochloride, methyl D-tyrosinate hydrochloride, H-D-tyrosine methyl ester-hydrochloride, D-tyrosine methyl ester hydrochloride, H-D-tyrosine methyl ester-hydrochloride, (2R)-2-amino-3-(4-hydroxyphenyl) propionic acid, (2R)-2-amino-3-(4-hydroxyphenyl) methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-tyrosine (3,5-I2)-OSu, Fmoc-tyrosine(3-N02)-OH, α-methyl-L-tyrosine, α-methyl-D-tyrosine, and α-methyl-DL-tyrosine. In certain embodiments of the invention, the tyrosine derivative is α-methyl-L-tyrosine as shown below:

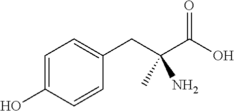

In other embodiments, the tyrosine derivative is α-methyl-D-tyrosine. In other embodiments, the tyrosine derivative is α-methyl-DL-tyrosine in a racemic form as shown below:

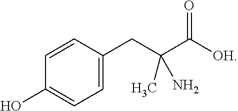

α-methyl-DL-tyrosine is also referred herein as DNP-01 or LI:79 or AMPT or α-methyl-para-tyrosine. In other words, the alternative names of α-methyl-DL-tyrosine include, for example, DNP-01, LI:79, AMPT, and α-methyl-para-tyrosine.

In a particular embodiment, the tyrosine derivative is a structural variant of α-methyl-L-tyrosine or α-methyl-DL-tyrosine. The structural variants of α-methyl-L-tyrosine or α-methyl-DL-tyrosine are well known in the art and fully described in, for example, U.S. Pat. No. 4,160,835, which is incorporated by reference herein in its entirety.

In one embodiment, the tyrosine derivative of the invention is an arylalanine compound having the formula:

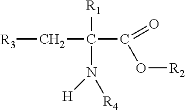

wherein R₁ is hydrogen, methyl or ethyl ester group, or alkyl of from 1 to 4 carbon atoms; R₂ is hydrogen, lower alkyl, lower alkene, succinimide, or alkyl of from 1 to 4 carbon atoms; R₃ is a substituted benzene ring of the following general formula

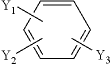

wherein Y₁, is located at the para position and is hydrogen, hydroxy, methyl ether, dimethyl ether, trimethyl ether, or an unsubstituted or halogen-substituted benzyl; Y₂, and Y₃ are the same or different and wherein one or both Y₂, and Y₃ located at either meta position or ortho position, and wherein Y₂, and Y₃ are hydrogen, hydroxy, halogen, methyl ether, or nitro; and R₄ is hydrogen, acetyl, tert-butyloxycarbonyl or fluorenylmethyloxycarbonyl.

In some embodiments, Y₁ and Y₂ are the same or different and are selected from hydrogen, cyanoamino, carboxyl, cyano, thiocarbamoyl, aminomethyl, guanidino, hydroxy, methanesulfonamido, nitro, amino, methanesulfonyloxy, carboxymethoxy, formyl, methoxy and a substituted or unsubstituted 5- or 6-membered heterocyclic ring containing carbon and one or more nitrogen, sulfur or oxygen atoms, specific examples of such heterocyclic rings being pyrrol-1-yl, 2-carboxypyrrol-1-yl, imidazol-2-ylamino, indol-1-yl, carbazol-9-yl, 4,5-dihydro-4-hydroxy-4-trifluoromethylthiazol-3-yl, 4-trifluoromethylthiazol-2-yl, imidazol-2-yl and 4,5-dihydroimidazol-2-yl, such that (a) Y1 and Y2 cannot both be hydroxy, (b) Y1 and Y2 cannot both be hydrogen and (c) when one of Y1 and Y2 is hydrogen, the other cannot be hydroxyl.

In one example, R₃ is a substituted or unsubstituted benzoheterocyclic ring having the formula:

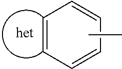

in which the benzoheterocyclic ring is selected from the group consisting of indolin-5-yl, 1-(N-benzoylcarbamimidoyl)-indolin-5-yl, 1-carbamimidoylindolin-5-yl, 1H-2-oxindol-5-yl, indol-5-yl, 2-mercaptobenzimidazol-5(6)-yl, 2-aminobenzimidazol-5(6)-yl, 2-methanesulfonamido-benzimidazol-5(6)-yl, 1H-benzoxazol-2-on-6-yl, 2-aminobenzothiazol-6-yl, 2-amino-4-mercaptobenzothiazol-6-yl, 2,1,3-benzothiadiazol-5-yl, 1,3-dihydro-2,2-dioxo-2,1,3-benzothiadiazol-5-yl, 1,3-dihydro-1,3-dimethyl-2,2-dioxo-2,1,3-benzothiadiazol-5-yl, 4-methyl-2(1H)-oxoquinolin-6-yl, quinoxalin-6-yl, 2-hydroxquinoxalin-6-yl, 2-hydroxyquinoxalin-7-yl, 2,3-dihydroxyquinoxalin-6-yl and 2,3-dihydro-3(4H)-oxo-1,4-benzoxazin-7-yl.

In another example, R₃ is a substituted or unsubstituted heterocyclic ring having the formula:

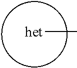

in which the heterocyclic ring is selected from the group consisting of 5-hydroxy-4H-pyran-4-on-2-yl, 2-hydroxypyrid-4-yl, 2-aminopyrid-4-yl, 2-carboxypyrid-4-yl, or tetrazolo[1,5-a]pyrid-7-yl.

In one particular embodiment, the tyrosine hydroxylase inhibitor is aquayamycin. In one example, aquayamycin is a compound of the formula set forth below.

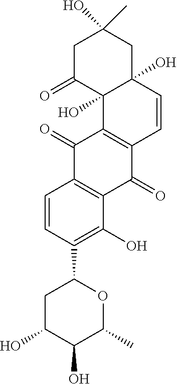 [SMILES: C[C@H]1O[C@@H](c2ccc3c(c2O)C(=O)C2=C(C3=O)[C@@]3(O)C(=O)C[C@](C)(O)C[C@@]3(O)C=C2)C[C@@H](O)[C@@H]1O]

In another particular embodiment, the tyrosine hydroxylase inhibitor is oudenone. In one example, oudenone is a compound of the formula set forth below.

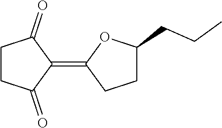

Other suitable tyrosine hydroxylase inhibitor, known to one of skilled in the art, can also be used. Example of other tyrosine hydroxylase inhibitor include, for example, but not limited to, cycloheximide, anisomycin, 3-iodo-L-tyrosine, pyratrione, phenyl carbonyl derivatives having catechol or triphenolic ring systems, for example, phenethylamine and gallic acid derivatives, 4-isopropyltropolone, 2-(4-thiazolyl)benzimidazole, 8-hydroxyquinoline, o-phenantroline, 5-iodo-8-hydroxyquinoline, bilirubin, 2,9-dimethyl-1, 10-phenantroline, α-α′-dipyridil, dibenzo [f,h]quinoxaline, 2,4,6-tripyridil-s-triazine, ethyl 3-amino4H-pyrrolo-isoxazole-5(6H)-carboxylate, α-nitroso-β-naphthol, sodium diethyldithiocarbamate, ethylenediamineteraacetic acid (See R Hochster, Metabolic Inhibitors V4: A Comprehensive Treatise 52 Elsevier (2012)).

In certain embodiments, the chewable pharmaceutical formulation comprises, for example, from about 5%, 10%, 20% 30%, 40%, or 50%, to about 60%, 70%, 80%, 90% or 95% tyrosine hydroxylase inhibitor by weight. For example, in some embodiments the chewable pharmaceutical formulation comprises at least about 5, 10, 15, 20, 25, 30, 35, 40, 45, 50, 55, 60, 65, 70, 75, 80, 85, 90, or 95% (w/w) of tyrosine hydroxylase inhibitor. In some embodiments, the concentration of tyrosine hydroxylase inhibitor in the chewable pharmaceutical formulation may range about 5-95, 10-80, 20-70, 25-65, 35-55, 40-50, 5-20, 10-30, 20-40, 30-50, 40-60, 50-70, 60-80, or 70-95% (w/w).

### Additives/Excipients for Enhancing Chewability

As used herein, to “enhance the chewability” of the provided pharmaceutical composition is defined as to improve and speed up the breakup, crumbling, crushing and/or disintegration of the ingested composition with or without mastication by the teeth in the mouth of the subject who has ingested, i.e., taken the composition into the mouth, and render the crushed particles into a soft, wet and palatable pulp and/or liquid in the mouth before the pulp and/or or liquid is swallowed, thereby releasing the tyrosine hydroxylase inhibitor, in particular α-methyl-DL-tyrosine, in the mouth faster than a release of the tyrosine hydroxylase inhibitor by dissolution of a “non-chewable” composition in the lower digestive tract, such as the stomach and intestines, of the subject. The “non-chewable” tablet with which the provided chewable pharmaceutical composition may be compared may be a tablet that is designed to be swallowed whole, i.e., it swells and breaks apart in the stomach, or the “non-chewable” tablet may be a tablet coated with an enteric coated tablet or capsule, i.e., it disintegrates in the small intestines, not the stomach. As used herein, “non-chewable” is defined as any tablet, which is formulated and designed for swallowing (without chewing, breaking or crushing), and moreover, if it is chewed the drug, a tyrosine hydroxylase inhibitor, will not be absorbed properly and may be ineffective, or may result in an overdose, e.g., if an enterically coated tablet (formulated for release in the small intestine,) is chewed it will release a large amount of the drug, rather than dissolving the drug over the intended amount of time.

To enhance the chewability of the herein provided chewable compositions comprising a tyrosine hydroxylase inhibitor, in particular α-methyl-DL-tyrosine, the tyrosine hydroxylase inhibitor is formulated with excipients which make the composition readily breakup and disintegrate in the mouth.

In various embodiments, herein provided chewable compositions are orally dissolvable and/or disintegrable, i.e., may be chewed by a person (i.e., a subject being treated with the chewable composition comprising a tyrosine hydroxylase inhibitor) who has ingested the chewable formulation into the mouth until substantially all of the ingredients contained therein substantially dissolve and disintegrate in the person's mouth. As used herein “substantially all” is defined as at least 50% up to 100% of the herein provided chewable compositions, e.g., of the chewable tablet, wafer, capsule, soft chew or gummy, is disintegrated in the subject's mouth, with chewing or without chewing, that is, is dissolved in the mouth without mastication by the subject who has ingested the chewable formulation. In various embodiments, the herein provided chewable compositions, “substantially all” is defined as at least 60%, at least 70%, at least 80%, at least 90%, at least 95%, at least 96% to 99%, or 100% of the of the herein provided chewable compositions is disintegrated in the subject's mouth, with chewing or without chewing (i.e., is dissolved in the mouth).

In various embodiments, the oral chewable formulation of a tyrosine hydroxylase inhibitor, particularly α-methyl-DL-tyrosine, is formulated with a excipients/additives selected from the group consisting of diluents, excipients, sticking agents, buffering agents, bulk agents, lubricating agents and colorants. Excipient bases for chewable compositions and formulations, such as tablets, wafers, and soft chewable compositions, including gummies and soft chews, are well known in the art and fully described in, for example, U.S. Pat. Nos. 9,808,010, 9,717,734, 9,474,713, and 5,686,107 and U.S. Patent Application Publications US2010/0278913, US2017/0360865, and US2010/0278913, which are incorporated by reference herein in their entirety. In certain embodiments, the oral chewable formulation of a tyrosine hydroxylase inhibitor, specifically α-methyl-DL-tyrosine may be a chewable tablet formulation. In other embodiments, the oral chewable formulation of a tyrosine hydroxylase inhibitor, specifically α-methyl-DL-tyrosine may be a chewable wafer. In alternate embodiments, the oral chewable formulation of a tyrosine hydroxylase inhibitor, in particular α-methyl-DL-tyrosine, may be a soft chewable composition, such as a soft chew or a gummie. Soft chewable composition are well known in the art and fully described in, for example, U.S. Patent Application Publication US 2017/0360865, U.S. Pat. Nos. 9,744,127, and 9,155,772 which are incorporated by reference herein in their entirety.

### Gum Core

In certain embodiments, the presently provided chewable formulations are chewable tablet formulations comprising a tyrosine hydroxylase inhibitor. In other embodiments, the presently provided chewable tablet formulations comprise α-methyl-DL-tyrosine (also called “alpha-methyl-para-tyrosine” or “AMPT” herein). In various embodiments, the chewable tablet formulations comprise an insoluble gum base core, which in certain embodiments may be coated, and in other embodiments may not be coated. In certain embodiments, the insoluble gum base core may comprise fillers, waxes, antioxidants, sweeteners, flavoring agents, and/or combinations thereof.

### Binders and Adhesives

A chewable tablet requires a tablet hardness that is acceptable for a chewable dosage form by being sufficiently hard to withstand processing and shipping, while also retaining a chewable texture. In certain embodiments, the provided chewable AMPT tablets may comprise a tablet binder as a major excipient. In various embodiments, binders (also called “binding agents”) that may be added to the formulations in dry granulation, include but are not limited, to microcrystalline cellulase (“MCC”), amylose, colloidal clays, and finely powdered acacia; such binders provide adhesion in slugging, i.e., the use of a tablet press for the compaction process. Solutions of polyvinylpyrrolidone (PVP), ethyl cellulose (EC), Hydroxypropyl Methyl Cellulose (HPMC) may be used as binding agents in the chewable tablet formulations provided herein when alcohol or other organic solvents are used, e.g., for a water-sensitive drug. In certain embodiments, PVP is the binder. In various embodiments, the chewable AMPT tablet formulations comprise as an excipient an aggregate of coprocessed excipients, including but not limited to microcrystalline cellulose and a galacotomannan, including but not limited to guar gum, locust bean gum, cassia gum, tara gum, or a mixture thereof, as is known and fully described in U.S. Pat. No. 5,686,107, which is incorporated by reference herein in its entirety. Co-processed excipients are a combination of two or more excipients designed to physically modify their properties in a way that is not achievable by simple physical mixing and without significant chemical change. Co-processed excipients have high functionalities compared to individual excipients, such as better flow property, compressibility and reduced lubricant sensitivity.

In certain embodiments, adhesives, which may be used for granulation, include but are not limited to, acacia, gelatin, liquid glucose, sucrose syrup, starch paste, methyl cellulose, carboxymethyl cellulose and mucilages of naturally occurring gums and colloidal clays.

In alternate embodiments, natural gums (also called biopolymers), such as xanthan gum, which is a polysaccharide, may be added as a binder and/or disintegrant, as well as a gelling agent. In some embodiments, gums/biopolymers that may be used as a binder, disintegrant and/or a gelling agent, include but are not limited to, alginate, chitosan, carrageenan, gellan gum, guar gum, gelatin, agar, alginate, carrageenan(s), such as iota carrageenan and kappa carrageenan, cellulose, gellan gum, gum Arabic, konjac gum, locust bean gum, modified starch, pectin and/or combinations thereof. In certain embodiments, the chewable tablet comprising AMPT includes at least one gum/biopolymer in an amount of about 1 wt. % to about 35 wt. %; in alternate embodiments, a gum/biopolymer is included in an amount of from about 2 wt. % to about 20 wt. %, e.g., from about 2 wt. % to about 5 wt. %, from about 5 wt. % to about 10 wt. %, from about 10 wt. % to about 15 wt. % or from about 15 wt. % to about 20 wt. %.

### Diluents/Fillers

Diluents, also called fillers, may be added to increase the bulk volume of a chewable tablet of AMPT. Combining, e.g., by mixing, a diluent with the active pharmaceutical ingredient, AMPT, the chewable tablet has a suitable weight and size for production and handling. A diluent will meet the all or most of the following requirement: being inert, biocompatible, non-hygroscopic, compactable, non-toxic, and non-conducive to microbiological growth/development.

Certain diluents are beneficial for chewable tablet formulation by compression and may be combined with AMPT in the provided chewable formulations include; these diluents include but not limited to mannitol, lactose, sucrose and sorbitol. These diluents assist in disintegration upon chewing and also help with acceptable taste and mouthfeel.

Additional diluents xylitol, dextrose, and starch, such as hydrolyzed starches. In certain embodiments, diluents that are sweet may be added to the chewable tablets as a sweetener excipient or as for the dual role of diluent and sweetener.

Mannitol may be added as a filler to the chewable tablet formulations comprising AMPT as the main excipient. Mannitol is a sugar alcohol, that increases blood glucose to a lesser extent than sucrose, and therefore, is used as a sweetener, particularly for diabetics. Mannitol is nonhygroscopic and demonstrates a low reactivity with drug substances. These properties make mannitol a suitable and advantageous diluent for chewable tablet comprising AMPT, as well as making it useful as a coating of chewable formulations. Mannitol has a pleasant sweet taste, as it is about 50% as sweet as sucrose, and has cooling effect in the mouth; in addition, it also has a smooth mouthfeel that is not gritty.

Mannitol-containing formulations typically comprise higher lubricant levels and higher glidant levels than other diluents/fillers for adequate compression to compensate for their poor flowing properties. However, a granular form of mannitol may be used as a direct-compression excipient. Mannitol may be added to chewable tablets when a rapid and complete solubility of the tablets is required.

In certain embodiments, sweeteners other than mannitol may be added to the chewable tablet AMPT as the main excipient, including but not limited to xylitol, sorbitol, sucrose, lactose, dextrose and hydrolyzed starches.

In various embodiments, xylitol may be added to a sugar-free oral chewable formulation of a tyrosine hydroxylase inhibitor, particularly α-methyl-DL-tyrosine, such as a chewable tablet formulation, comprising AMPT. Xylitol is sweeter than mannitol, and also has a cooling effect. Xylitol may be used in the herein provided chewable formulations for its non-acidogenic property, which does not promote tooth decay.

In certain embodiments, sorbitol is added to the chewable tablet formulations comprising AMPT. Sorbitol, an isomer of mannitol, is slightly sweeter than mannitol and is hygroscopic at humidity above 65%, and thus may clump in the feed system and stick to the die table when tableted during manufacturing. Sorbitol is commercially available as Sorb-Tab and crystalline Tablet Type for direct compression. Sorbitol produces a tablet that is harder than a mannitol comprising chewable tablet. Sorbitol does have a laxative effect; therefore, it may be added in a low concentration, i.e., a lower concentration than mannitol, such as from 1-50 wt. %. In alternate embodiments, sorbitol is included in an amount of from about 1 wt % to about 40 wt. %, e.g., from about 2 wt. % to about 5 wt. %, from about 5 wt. % to about 10 wt. %, from about 10 wt. % to about 15 wt. %, from about 15 wt. % to about 20 wt. %, from about 20 wt. % to about 25 wt. %, from about 25 wt. % to about 30 wt. %, from about 30 wt. % to about 35 wt. % or from about 35 wt. % to about 40 wt. %.

Lactose is a widely used excipient, however, since it has a low level of sweetness of about 15% (w/w) of sucrose, artificial sweeteners may be added to enhance its taste. Lactose may be used in its anhydrous powder form for direct compression, since it has good flow and compressibility properties. In wet granulation, hydrous lactose powders may be used as either a 60-80 mesh (course) grade or an 80-100 mesh (regular pharmaceutical) grade. Anhydrous lactose may discolor certain drugs, i.e., amine drug bases or salts of alkaline compounds; anhydrous lactose absorbs moisture in humidity. For those subjects who are lactose intolerant, a lactose diluent will be unsuitable. Microcrystalline cellulose (AVICEL®), a purified partially depolymerized cellulose, may be used as a filler, in addition to its use as a dry binder and as a disintegrant.

### Disintegrants

A disintegrant (also called a dissolution enhancer) may be added to the chewable tablet formulations provided herein to enable tablet break up (disintegration) when the tablet is in contact with fluids of the gastrointestinal tract and promote fast drug dissolution. A dissolution enhancer alters the molecular forces between chewable formulation ingredients to increase the dissolution of solute in the solvent (such as one or more of saliva, gastric acid, intestinal juice, bile, pancreatic juice). Disintegration of the chewable tablets is critical for bioavailability of the AMPT. Disintegrants are hygroscopic and absorb fluids into the tablet matrix; they act by either facilitating water uptake and causing the tablet to break into fragments or by rupturing the tablet by swelling of the disintegrant particles during fluid absorption. Starch, a traditional disintegrant, draws water into the tablet by capillary action; the spherical starch grain shape increases tablet porosity thereby enabling wicking of liquid into the tablet.

In certain embodiments, a disintegrant that may be added to the provided chewable tablets formulations comprising AMPT, includes but is not limited to, starch (includes starch from corn, potato, wheat) and starch derivatives, such as sodium starch glycolate (commercially available as EXPLOTAB®, VIVASTAR® and PRIMOGEL®), cellulose (microcrystalline cellulase (“MCC”), commercially available as AVICEL® PH101 and AVICEL® PH102) and sodium carboxymethyl cellulose (Na-CMC) (and a combination thereof commercially available as RC 591 from SANCEL®) and cellulose derivatives, such as croscarmellose sodium (a crosslinked Na-CMC, commercially available as Ac-Di-Sol®), crosslinked polymers, such as crosslinked polyvinylpyrrolidone (PVP) [also called crospovidone], clays, such as bentonite, alginates, and a cation exchange resin. In various embodiments, the provided chewable tablets formulations comprising AMPT comprise a disintegrant, such as fructose, povidone (polyvinylpyrrolidone (PVP)), a surfactant or combinations thereof.

In other embodiments, a disintegrant that may be added to the provided chewable tablets formulations comprising AMPT, includes but is not limited to a natural gum (also called biopolymer), such as xanthan gum, alginate, chitosan, carrageenan, gellan gum, guar gum, gelatin, agar, alginate, carrageenan(s), such as iota carrageenan and kappa carrageenan, cellulose, gellan gum, gum Arabic, konjac gum, locust bean gum, modified starch, pectin and/or combinations thereof. In certain embodiments, the chewable tablet comprising AMPT includes at least one gum/biopolymer in an amount of about 1 wt % to about 35 wt %; in alternate embodiments, a gum/biopolymer is included in an amount of from about 2 wt % to about 20 wt %, e.g., from about 2 wt % to about 5 wt %, from about 5 wt % to about 10 wt %, from about 10 wt % to about 15 wt % or from about 15 wt % to about 20 wt %.

In certain embodiments, a wetting agent, also called a surfactant, may be added to the herein provided chewable formulations to aid in uptake of water by the formulation, to thereby enhance disintegration and aid in drug dissolution (AMPT). A surfactant decreases the surface tension between two liquids or between a liquid and a solid, hence increasing the solubility of the solubility. Starch combined with a surfactant, such as the anionic surfactant sodium lauryl sulphate (SLS), in a dry state provides faster disintegration and dissolution rates than starch treated with a solution of surfactant. Starch treated with Polysorbate 80, a nonionic surfactant, exhibits a better dissolution profile than SLS-treated starch. In certain embodiments, starches may be combined with or treated with a surfactant, including but not limited to SLS and Polysorbate 80. In other embodiments, a cationic surfactant, such a Cetrimid (an antiseptic which is a mixture of different quaternary ammonium salts, including cetrimonium bromide), or a nonionic surfactant, such as sorbitan fatty acid esters (Spans), e.g., Span® 80, and/or polyethoxylated sorbitan esters (Tweens, which are ethoxylated Spans) may be added to the chewable formulations provided herein. In various embodiments Spans and Tweens are solubilizers, dispersing agents and wetting agents.

In some embodiments, a disintegrant may be added to the provided chewable tablet formulation in a concentration of up to about 20% (w/w). In certain embodiments, a disintegrant may be added to the chewable tablet formulation in a concentration of up to about 10% (w/w). In alternate embodiments, a disintegrant may be added to the chewable tablet formulation in a in an amount of from about 1 wt % to about 35 wt %, or from about 2 wt % to about 20 wt %, e.g., from about 2 wt % to about 5 wt %, from about 5 wt % to about 10 wt %, from about 10 wt % to about 15 wt % or from about 15 wt % to about 20 wt %.

Sodium starch glycolate, a modified starch, swells 7- to 12-fold in less than 30 seconds, and is used as a super-disintegrant. Croscarmellose sodium, a modified cellulose, swells 4- to 8-fold in less than 10 seconds, also is used as a super-disintegrant, facilitating fast tablet breakup and dissolution in the intestinal tract after oral administration; its crosslinking allows enhanced bioavailability of the drug through superior drug dissolution. In certain embodiments, super-disintegrants, including but not limited to sodium starch glycolate and croscarmellose sodium may be added to the chewable tablet formulations in a low concentration of from about 1% to about 8% (w/w). In other embodiments, a super-disintegrant may be added to the chewable tablet formulations in a concentration of from about 1% to about 5% (w/w). In alternate embodiments, a super-disintegrant may be added to the chewable tablet formulations in a concentration of from about 1% to about 4% (w/w). In certain embodiments, a super-disintegrant may be added to the chewable tablet formulations in a concentration of from about 1% to about 3% (w/w). In various embodiments, a super-disintegrant may be added to the chewable tablet formulations in a concentration of: about 5% (w/w), about 4% (w/w), about 3% (w/w), about 2% (w/w), or about 1% (w/w).

### Lubricants, Anti-adherants and Glidants

In various embodiments, the provided chewable tablets comprise a lubricant, which may be a water-insoluble (fatty acid-based) lubricant or a water-soluble lubricant. A lubricant reduces friction during tablet formulation in a die, as well as during ejection from the die cavity. Water-insoluble lubricants, include but are not limited to, magnesium stearate, calcium stearate, stearic acid, stearic acid salt, talc, silica derived-colloidal silica, such as CAB-O-SIL® (fumed silicas available with either hydrophobic or hydrophilic surfaces commercially available from Cabot Corporation) and several powder colloidal silicon dioxide AEROSIL® products and AEROPERL® granulate colloidal silicon dioxide product (both commercially available from Evonik), liquid paraffin and propylene glycol. Water-soluble lubricants, include but are not limited to, polyethylene glycol, sodium chloride, and magnesium/sodium lauryl sulfate.

In certain embodiments, an anti-adherant that may be added to the chewable tablet formulations. An anti-adherant reduces sticking or adhesion of the tablet granulation or powder to the faces of a tablet punch or to a die wall. Anti-adherents, include but are not limited to, talc, cornstarch and sodium dodecylsulfate.

In other embodiments, a glidant that may be added to the provided chewable tablet formulations. A glidant improves the flow of the tablet granulation or powder mixture from a hopper to a die cavity by decreasing inter-particulate friction between the particles. Glidants also prevent powder caking, optimize tablet weight uniformity and improve the mechanical tablet stability. A glidant may be added during direct compression and to granulation before tableting. Glidants, include but are not limited to, fine silica, talc, corn starch, colloidal silica and magnesium stearate. In certain embodiments, a colloidal silicon dioxide (such as AEROSIL® 200 Pharma, AEROSIL® 200 VV Pharma, AEROSIL® 300 Pharma and AEROSIL® R 972 Pharma) may be used as a glidant.

### Taste Masking Agents: Flavors, Sweeteners, Lipids and Acids

In formulating chewable AMPT tablets, issues of sweetness, chewability, mouthfeel (physical sensations in the mouth caused by food/drink, also called “texture”) and taste must be taken into consideration. Principal excipients, such as fillers or direct-compression vehicle, have an important function in the outcome of these issues. Sweeteners contribute relevant features to chewable tablets, namely, sweetness and chewability.

The unpalatable taste of the drug AMPT may be reduced by adding taste masking agents, such as flavors (also called “flavoring agents” or “flavorant”), sweeteners and/or effervescent agents, such as sodium bicarbonate and citric acid. Another way to mask the unpleasant taste is by preventing contact of the bitter/unpleasant drug with the taste buds by a formulating the chewable tablet with a bitterness blocking agent to mask the bitter taste or the perception of bitter on the tongue, including but not limited to adenosine monophosphate, lipoproteins, or phospholipids. The addition of sodium chloride to a formulation also masks bitterness of the drug.

Another method for taste-masking is coating the drug particles with coating compositions that are insoluble in the mouth, including but not limited to hydrophobic or hydrophilic polymers, lipids, as well as sweeteners, alone or in combination, to produce a single or multi-layer coat. Alternative effective taste-masking coating compositions, include but are not limited to, polymers, such as methacrylic acid and methacrylic ester copolymers, e.g., Eudragit E-100, RL 30D, RS 30D, L30D-55, and NE 30D. The polymer coat levels for taste-masking may vary from 10% to 40% depending on the drug bitterness.

An alternate formulation method for taste-masking is depositing successive layers of an active compound onto inert starter seeds, such as sugar spheres or microcrystalline cellulose (MCC) speres, such as CELPHERE™ (commercially available from Asahi Kasei Corporation). The bitter drug may be dissolved or dispersed in an aqueous or non-aqueous solvent along with a binder to permit the drug particles to adhere to the inert substrate. In certain embodiments a binder includes, but is not limited to, hydroxypropyl methylcellulose (HPMC), hydroxypropyl cellulose (HPC), povidone, Eudragit E-100, and carboxymethyl cellulose. The drug-layered beads may subsequently be coated with a taste-masking polymer that delays drug dissolution in the oral cavity. In some embodiments, a polymer used for taste-masking purposes includes but is not limited to Eudragit E-100, ethylcellulose, HPMC, HPC, polyvinyl alcohol, and polyvinyl acetate. The taste-masked coated beads may then be incorporated into the final dosage form, such as a compressed chewable tablet or a chewable capsule.

A further formulation method for taste-masking is granulating the drug, followed by coating the drug-loaded granules with a taste-masking polymer (“a granulation-coat approach”). Granulation decreases the surface area of the drug by increasing its particle size, thereby minimizing the amount of taste-masking polymer required. A granulation-coat approach may be used over a layer-coat for high doses, since the granulation process may provide high drug loading. Fluid-bed coating is an effective industrial process for applying a polymer coat for taste-masking; coated particles after such fluid-bed coating generally withstand the tablet compression process used to manufacture the final dosage form (chewable tablet).

An additional method for taste-masking is a hot-melt extrusion process, in which the bitter active (the drug AMPT) is mixed with other ingredients in a dry state without added organic solvents, followed by filling the mixture in a hopper, conveying, mixing, and melting by an extruder. The ingredients are heated under intense mixing to obtain the taste-masked extrudates. The extrudate may then be milled or micronized to obtain taste-masked granules or particles, which subsequently may be incorporated into a suitable dosage form. Twin screw extruders provide advantages, such as short transit time, convenient material feed, high shear kneading, and less over-heating.

Three further methods for taste-masking oral dosage forms of the provided chewable tablet formulations are microencapsulation, complexation and spray-drying. A microencapsulation process is used to encapsulate the bitter active pharmaceutical ingredient (API), AMPT, thus preventing its contact with taste buds. Microcaps® is an example of microencapsulation technology that applies coacervation/phase separation to produce different encapsulated polymeric membranes; the process mainly consists of formation of three immiscible phases, formation of the coat, and deposition of the coat. The formation of the three immiscible phases is achieved by dispersing the core particles in a polymer solution. A phase separation is then induced by change in the temperature of polymer solution; change in the pH, addition of a salt, non-solvent, or by inducing a polymer-polymer interaction. The phase separation leads to deposition of the polymer coat on the core material under constant stirring. Next, the core particles coated by the polymer are separated from the liquid phase by thermal, crosslinking, or desolvation methods to render the coat rigid. Microcaps® technology is used in combination with Advatab® (from Adare Pharmaceuticals) compressed orally-disintegrating tablet (“ODT”) technology.

A complexation process is used for taste-masking bitter drugs by forming inclusion complexes of cyclodextrins with the drug molecule. Cyclodextrins are distinctive bucket-shaped cyclic oligosaccharides containing at least six D-(+)-glucopyranose units attached by alpha-(1,4)-glucosidic bonds with a molecular structure of hydrophobic cavity and hydrophilic exterior. The formation of inclusion complexes and its type depends on several factors like drug properties, processes involved, the equilibrium kinetics, formulation excipients, and the desired final dosage form and delivery system. Taste-masking is attained by the interaction of cyclodextrins with proteins of the taste buds or by inhibition of contact between bitter drug molecules and taste buds.

An alternative to using cyclodextrins is to use ion exchange resins to achieve taste-masking by complexation; ion exchange resins are high molecular weight polymers with cationic and anionic functional groups. This complexation process involves suspending the resin in a solvent in which the drug (AMPT) is dissolved to form a drug-resin complex, known as a “drug-resinate”, which prevents direct contact of the drug with taste buds, thereby providing taste-masking during administration of the drug-resinate. Once ingested, the resin exchanges the drug with the counter ion in the gastrointestinal tract, and the drug is released to be absorbed. Commercially available ion exchange resins that may be used for taste-masking are based on methacrylic acid-divinyl benzene polymer and styrene-divinyl benzene polymer.

A spray-drying process offers another approach to taste-masking by applying a physical barrier coating. The bitter drug is either dissolved or dispersed together with the coating polymer in a suitable solvent followed by spray-drying. Typically, this process consists of three different steps: (1) atomization of feed into a spray, (2) spray-air contact (mixing and flow) followed by drying, and (3) separation of dried product from the air. The process permits the selection of aqueous or non-aqueous solvents. The dried product frequently includes granules or beads containing taste-masked encapsulated drug. The amount of polymer coat may delay the drug release, and therefore requires careful polymer selection and process design to afford taste-masking. Moreover, the formulation and processing may affect whether the polymer is “coated” on the surface or dispersed. Superior taste-masking is determined by providing a coat, not a dispersion. Some advantages of spray-drying include: (a) less processing time, as it is a single step process, (b) scale-up capability, and (c) a wide variety in the choice of solvent and polymer.

### Flavoring Agents

The taste of a chewable tablet is an important consideration for consumer/patient acceptance of such a tablet. The perceptions of mouth-feel, sweetness and flavor combined in taste sensation.

Mouth-feel is affected by heat of solution of the soluble components, smoothness of the combination during chewing, and hardness of the tablet. These factors are directly and almost completely related to the active ingredient and major excipients. Sweetness, at an appropriate level, is a necessary background to any flavor. The primary contributors to sweetness in a chewable tablet are the drug, natural sweetener(s) and artificial sweetener(s) that may be incorporated in the formulation. Flavoring agents are well-known to the ordinarily skilled artisan and are available in a variety of physical forms from commercial suppliers specializing in these materials. Flavors may be added to improve the taste of the herein provided chewable tablets, as well as of mouth dissolved tablets. Various flavoring agents forms available include water-miscible solutions, oil bases, emulsions, dry powders, spray-dried bead lets, and dry adsorbates. A typical flavor has the capability of producing several hundred combinations for a given formulation application.

A flavoring agent may be added to the herein provided chewable formulations to improve the taste of the active pharmaceutical ingredient, such as AMPT. The five basic tastes are salty, sweet, bitter, sour and savory (umami). In certain embodiments, flavoring agents that may be added to the chewable AMPT tablets for taste types include sweet, sour (acidic), salty, savory and bitter. Sweet flavoring agents include, but are not limited to, honey, berry, grape and vanilla flavor. Sour flavoring agents include, but are not limited to, cherry, strawberry, citrus (such as orange) and liquorice flavor. Salty flavoring agents include, but are not limited to, spice, mixed fruit, mixed citrus and buttery flavor. Bitter flavoring agents include, but are not limited to, mint, liquorice, nut, fennel, grapefruit and wine flavor. Savory flavoring agents include, but are not limited to, tomato, mushroom (shiitake), meaty, soy, fermented products (such as smoked or fermented fish, cheese, barley and soy) and glutamate-, inosine monophosphate- and guanosine monophosphate-containing food (including but not limited to monosodium glutamate and umami tastes, such as green tea, yeast extracts), and L-aspartate (aspartic acid) flavor.

Flavoring agents are frequently thermolabile, and thus, may not be added before an operation in the tablet manufacturing process that involves heat. Flavors may be incorporated either as solids (spray dried flavors) or oils or aqueous (water soluble) flavors. Flavoring agents may be mixed with the granules as an alcohol solution. A solid dry flavor is easier to handle and commonly more stable than oils. An oil flavor is usually added at the lubrication step because of its sensitivity to moisture and its tendency to volatilize when heated during drying. A flavor also may be adsorbed onto an excipient and added during the lubrication process. The maximum amount of oil that can be added to granulation without affecting tableting characteristics is 0.5 to 0.75% w/w. Aqueous flavors typically are less used because of their instability on aging.

### Sweeteners

Sweeteners and sweetener compositions are well known in the art and fully described in, for example, U.S. Patent Application Publication US2017/0354175, which is incorporated by reference herein in its entirety. A sweetener may be added to the chewable AMPT tablet formulations provided herein either to exclude or to limit the addition of sugar in the formulations. In certain embodiments, blends of sweeteners may be added to the herein provided chewable tablet formulations, such as a blend of artificial sweeteners, a blend of natural and artificial sweeteners, or a blend of natural sweeteners. The chewable tablet formulations comprising AMPT may include a sweetener, which provides not only the required “sweetness” property, but also the “chewability” characteristic from the sweetener itself being chewable. In certain aspects, natural sweeteners, such as mannitol, lactose, sucrose, and/or dextrose may be added as a sweetener to the chewable formulations provided herein. In various embodiments, artificial sweeteners, such as aspartame, neotame, saccharin, cyclamate, may be used in place of, or in addition to, natural sweeteners. In alternate embodiments, the chewable formulations provided herein exclude saccharin and cyclamate as a sweetener.

In certain embodiments, the chewable AMPT formulations may include, but do not limit, the sweetener to small molecule saccharides, such as honey and/or high fructose corn syrup, monosaccharides, such as fructose, glucose, and xylose, disaccharides, such as sucrose, trehalose, and lactose, trisaccharides, polysaccharides, oligosaccharides, such as fructan and inulins, sugar alcohols, such as sorbitol, xylitol, lactitol, and maltitol, and mixtures of sugars, such as combinations of one or more of honey, corn syrups, light corn syrups and/or high fructose corn syrups.

Additional non-limiting examples of a sweetener that may be added to the provided AMPT chewable formulations, include but are not limited to stevia, monk fruit sugar, agave syrup, crystalline fructose, high fructose corn syrup, tapioca syrups, sucralose, sorbitol, xylitol, and combinations thereof. In certain embodiments, the chewable tablet formulations comprising AMPT may include natural or artificial sweeteners, sugar alcohol, or other sugar substitute in place of all or part of its sucrose. While saccharin is from 300 to 500 times as sweet as sucrose, it has a bitter after-taste; accordingly, any of the herein described taste-masking agents may be added to the chewable formulations provided herein comprising saccharin or any of the herein-described methods to taste-mask may be used to decrease, mask, coat and/or block such bitter flavors. Aspartame is about 180 to 200 times sweeter than sucrose, however, aspartame lacks stability in the presence of moisture; thus, chewable formulations provided herein comprising aspartame may be formulated in low moisture environmental conditions and/or formulated with non-hygroscopic excipients. In alternate embodiments, the artificial sweetener may be neotame. In other embodiments, the artificial sweetener may be advantame (commercially available from Ajinomoto Co.), a non-caloric artificial sweetener synthesized from isovanillin and aspartame, is about 20 thousand time as sweet as sucrose; it is classified as generally recognized as safe (“GRAS”).

In various embodiments, a sweetener may be included in an amount of from about 2 wt. % to about 60 wt. %, or alternatively, in an amount of from about 5 wt. % to about 30 wt. %, such as from about 5 wt. % to about 25 wt. %, from about 10 wt. % to about 20 wt. %, from about 15 wt. % to about 20 wt. %, from about 20 wt. % to about 25 wt. % or from about 15 wt. % to about 30 wt. %.

In other embodiments, the oral chewable formulation of a tyrosine hydroxylase inhibitor, particularly α-methyl-DL-tyrosine, may be sugar-free. In various embodiments, the oral chewable formulation may be a chewable tablet formulation comprising AMPT.

In alternate embodiments, the chewable tablet formulations comprising AMPT may comprise from about 0.001% to about 1% sucralose (or any known artificial sweetener), alternatively from about 0.01% to about 0.5% sucralose (or other artificial sweetener), alternatively from about 0.03% to about 0.1% sucralose (or other artificial sweetener).

TABLE 1
Estimated Relative Sweetness of Various Sweeteners

| Sweetener Name | Relative Sweetness (relative to sucrose) |
|---|---|
| Neotame | 7,000-13,000 |
| Aspartame (Methyl L-α-aspartyl-L- phenylalaninate) | 180-200 |
| Saccharin Sodium (benzoic sulfimide) | 300-500 |
| Glycyrrhizin (glycyrrhizic acid or glycyrrhizinic acid) from Glycyrrhiza glabra (liquorice) root | 50 |
| Sucrose (Sugar) | 1 |
| Sorbitol (D D-glucitol) | 0.5-0.6 |
| Mannitol | 0.5-0.7 |
| Dextrose (D-Glucose) | 0.7 |
| Maltose (4-O-α-D-Glucopyranosyl- D-glucose) | 0.3 |
| Fructose (fruit sugar/levulose) | 1.7 |
| Lactose (Milk sugar/ 4-O-β-D-galactopyranosyl- D-glucose) | 0.2 |

### Coloring Agents

Coloring agents (also called colorants) may be added to mask the color of the drug AMPT, to identify the drug product and/or to produce a more elegant look, i.e., an esthetically appealing, chewable tablet product. In embodiments, the color of the chewable tablet has a matching flavoring agent (orange shade of color with the characteristic sweet-sour taste of orange-flavor). Likewise, the aroma of the chewable tablet formulation corresponds to the flavor (orange aroma for characteristic sweet-sour taste of orange-flavor).

All coloring agents are approved by the United States Food and Drug Administration (FDA) as being acceptable for use in humans or domestic animals. Approved colorants permitted by legislative bodies and/or regulatory agencies may vary from country to country. Two forms of colors are used in tablet preparation: FD & C and D & C approved dyes. Dyes are water-soluble and are applied as a solution in a dry granulation mix or in a vehicle for wet granulation. Wet granulation with water-soluble dyes provides a better color uniformity, but dye migration to the top of granules together with solvent during drying may arise. Water-soluble dyes also may be adsorbed into a carrier, such as starch or lactose, and dry blended before a final mix. Water-insoluble pigments may be used in direct compression and are dry blended with other tablet components; such water-insoluble pigments, such as iron oxides, titanium dioxide, and some aluminum lakes, also may provide opacity to a tablet coating.

Lakes are dyes formed by absorption on hydrous oxide, such as aluminum hydroxide, resulting in an insoluble form of the dye; lake dyes are employed as dry powders for coloring in dry granulation. FD&C dyes that may be used in the herein provided chewable formulations as a coloring agent include, but are not limited to, FD & C Blue 2 (Indigo carmine or indigotine), FD & C Blue 1 (brilliant blue), FD & C Green 3 (fast green, which is a bluish green or aqua color), FD & C Yellow 5 (tartrazine), FD & C Yellow 6 (sunset yellow/orange yellow), FD&C Red 3 (erythrosine) and FD&C Red 40 (Allura red AC). Examples of FD&C aluminum lakes that may be uses as a coloring agent include, but are not limited to, FD&C Blue 1 Aluminum Lake, FD&C Red 40 Aluminum Lake, FD&C Yellow 5 Aluminum Lake, and FD&C Yellow 6 Aluminum Lake. Lakes may be used in coloring tablet coatings, since they are more stable than and have a greater opacity than water-soluble dye.

Dyes are dissolved in the granulating system to be incorporated during the granulation process. Colorants may be added before compaction as an insoluble powder or dissolved in the granulation liquid. Colors at either extreme of the visible spectrum, blue and red, show mottling, while colors near the mid-range of the spectrum show less spotting. Chewable colored tablet formulations typically may be checked for resistance to color changes on exposure to light or fading.

### Adsorbents

In certain embodiments, an adsorbent may be added to the herein provided chewable formulations. Adsorbents are agents that retain large quantities of liquids. Thus, for example, vitamin E (tocopherols), essential oils, and hygroscopic agents may be incorporated into the herein provided chewable tablet formulations by adding an adsorbent. Typically, the liquid to be adsorbed is first mixed with an adsorbent before incorporation into the chewable formulations. An adsorbent, includes but is not limited to anhydrous calcium phosphate, starch, magnesium carbonate, bentonite, kaolin, magnesium silicate, magnesium oxide and silicon dioxide (also called colloidal silicon dioxide), such as various AEROSIL® colloidal silicon dioxides (also used as a glidant).

### Preservatives and Antioxidants

In various embodiments, a preservative may be added to the herein provided chewable formulations. A preservative, includes but is not limited to, parabens, such as methyl, propyl, benzyl, butyl p-hydroxy benzoate.

In some embodiments, the herein provided chewable formulations comprise an antioxidant. An antioxidant, includes but is not limited to ascorbic acid and their esters, alpha-tocopherol, ethylene diamine tetra acetic acid, sodium metabisulfite, sodium bisulfite, Butylated Hydroxy Toluene (BHT), Butylated Hydroxy Anisole (BHA), citric acid, and tartaric acid. In other embodiments, a chelating agent (also called a chelator) is added to the herein provided chewable formulations as an antioxidant; a chelator includes but is not limited to ethylenediamine tetraacetic acid and its salts, dihydroxy ethyl glycine, citric acid and tartaric acid.

In certain embodiments, the herein provided chewable formulations may be preservative-free, flavor-free and/or artificial color-free.

### Alkalinizing Agents

It is well-known and fully described in, for example, U.S. Pat. No. 4,165,382, which is incorporated by reference herein in its entirety, that treatment with alpha-methyl-para-tyrosine results in crystals in the urine (crystalluria). To avoid or abolish crystalluria, an alkalinizing agent may be included as component of the chewable tablet, or may be taken as a separately administered agent (product) concurrently with the chewable tablet, in an amount sufficient to cause the urine of the person treated with alpha-methyl-para-tyrosine to have an alkaline pH (basic), such as a pH above about 7.4.

An alkalinizing agent (also called a buffer or buffering agent) includes, but is not limited to, sodium bicarbonate, ammonium chloride, calcium carbonate (commercially available as TUMS and ROLAIDS), sodium citrate/citric acid (commercially available as Cytra-2 and Virtrate-2), potassium citrate/citric acid (Virtrate-K), tricitrates (citric acid, potassium citrate [also known as tripotassium citrate] and sodium citrate, available as Cytra-3, Virtrate-3, Polycitra, Polycitra-LC oral syrup).

In various embodiments, at least one alkalinizing agent is added to the chewable AMPT tablet formulation as an admixed and/or granulated component in an amount effective to make the urine of the subject being treated with the chewable AMPT tablet formulation alkaline. In various embodiments, the urine of the treated subject is rendered a pH of greater than about 7.4. In other embodiments, the urine of the treated subject is rendered a pH of about 7.8 to about 8.0. In certain embodiments, the at least one alkalinizing agent may be selected from sodium bicarbonate, ammonium chloride, calcium carbonate, a sodium citrate/citric acid combination, a potassium citrate/citric acid combination, and tricitrates (a combination of citric acid, potassium citrate and sodium citrate).

In alternative embodiments, the at least one alkalinizing agent is not a component of the chewable AMPT tablet formulation, but the alkalinizing agent is administered as a separate product that commercially available, e.g., TUMS, Cytra-3, and/or a comparable alkalinizing agent effective to cause the urine of the subject treated with the chewable AMPT tablet formulation basic. In an embodiment, the urine is rendered a pH of greater than about 7.4. In alternate embodiments, the urine of the treated subject administered an alkalinizing agent separately from the administered chewable AMPT tablet formulation is rendered a pH of about 7.8 to about 8.0.

Effective amounts of chewability enhancing excipient(s) may be determined by routine experimentation informed by the guidance provided herein. For example, in some embodiments, the chewable pharmaceutical formulation comprises at least about 5, 10, 15, 20, 25, 30, 35, 40, 45, 50, 55, 60, 65, 70, 75, 80, 85, 90, or 95% (w/w) of the chewability enhancing excipient(s). In some embodiments, the concentration of the chewability enhancing excipient(s) in the pharmaceutical formulation may range about 5-95, 10-80, 20-70, 25-65, 35-55, 40-50, 5-20, 10-30, 20-40, 30-50, 40-60, 50-70, 60-80, or 70-95% (w/w).

### Additional Therapeutic Agents

Pharmaceutical compositions and kits comprising a tyrosine hydroxylase inhibitor and a second therapeutic agent are well known in the art and fully described in, for example, U.S. Pat. Nos. 9,895,425, and 9,763,903, which are incorporated by reference herein in their entirety.

The herein provided oral chewable formulations comprising a therapeutically effective amount of a tyrosine hydroxylase inhibitor, particularly α-methyl-DL-tyrosine, may further comprise a therapeutically effective amount of one or more another therapeutic agent. In certain embodiments, the one or more another therapeutic agent is an antidepressant, a benzodiazepine, a glucocorticoid, a cannabinoid or a combination thereof. In some embodiments, the one or more another therapeutic agent is a vasopressin analog. In other embodiments, the vasopressin analog is desompressin.

In alternate embodiments, the one or more another therapeutic agent is a neuromodulating agent. In various embodiments, the neuromodulating agent is γ-aminobutyric acid (GABA). In certain embodiments, the neuromodulating agent potentiates acetylcholine. In other embodiments, the neuromodulating agent is rivastigmine, or pilocarpine, or similar agents.

In particular embodiments, the tyrosine hydroxylase inhibitor is racemic α-methyl-DL-tyrosine and said one or more another therapeutic agent comprise desompressin and GABA. As used herein, “another therapeutic agent” is one or more therapeutic agent other than a tyrosine hydroxylase inhibitor. In alternate embodiments, the one or more another agent comprises GABA. In certain embodiments, the antidepressant is a selective serotonin reuptake inhibitor (SSRI), a serotonin-norepinephrine reuptake inhibitor (SNRI), a tricyclic antidepressant, or a combination thereof. In various embodiments, the antidepressant is sertraline, fluoxetine, paroxetine, venlafaxine, or a combination thereof.

In additional embodiments of the herein provided oral chewable formulations comprising a therapeutically effective amount of a tyrosine hydroxylase inhibitor, particularly α-methyl-DL-tyrosine, the formulations further comprise a therapeutically effective amount of a melanin promoter; a p450 3A4 promoter; and a leucine aminopeptidase inhibitor, wherein the melanin promoter is methoxsalen or melanotan II; the p450 3A4 promoter is 5,5-diphenylhydantoin, valproic acid, or carbamazepine; and the leucine aminopeptidase inhibitor is N-[(2S,3R)-3-amino-2-hydroxy-4-phenylbutyryl]-L-leucine or rapamycin, as is well known in the art and fully described in U.S. Pat. No. 9,895,425, which is incorporated by reference herein in their entirety. In particular embodiments, oral chewable formulations comprising a therapeutically effective amount of a tyrosine hydroxylase inhibitor, particularly α-methyl-DL-tyrosine, the formulations further comprise a therapeutically effective amount of a melanin promoter; a p450 3A4 promoter; and a leucine aminopeptidase inhibitor, wherein the melanin promoter is methoxsalen or melanotan II; the p450 3A4 promoter is 5,5-diphenylhydantoin, valproic acid, or carbamazepine; and the leucine aminopeptidase inhibitor is N-[(2S,3R)-3-amino-2-hydroxy-4-phenylbutyryl]-L-leucine or rapamycin, and GABA. In alternate embodiments, the herein provided oral chewable formulations comprising a therapeutically effective amount of a tyrosine hydroxylase inhibitor, particularly α-methyl-DL-tyrosine, further comprise a growth hormone inhibitor.

In certain embodiments, the one or more another therapeutic agent is a beta adrenergic agonist (also referred to as beta agonists). In various embodiments, beta adrenergic agonist is albuterol, levalbuterol, fenoterol, formoterol, isoproterenol, metaproterenol, salmeterol, terbutaline, clenbuterol, isoetarine, pirbuterol, procaterol, ritodrine, epinephrine, and combinations thereof, as is well known in the art and fully described in U.S. Pat. No. 9,895,425, which is incorporated by reference herein in their entirety.

In additional embodiments the one or more another therapeutic agent is an autonomic neurotransmission (e.g. amphetamine, methylphenidate, and the like), a psychotopic drug (e.g., risperidone), a neutotransmitter reuptake inhibitor (e.g., fluoxetine), a compound that stimulates glutaminergic transmission (e.g., LY2140023), and/or a compound that affects cholinergic neurotransmission (e.g., galantamine), as is well known in the art and fully described in U.S. Pat. No. 9,895,425, which is incorporated by reference herein in their entirety.

The invention also provides a pharmaceutical composition comprising compounds of the invention and one or more pharmaceutically acceptable carriers. “Pharmaceutically acceptable carriers” include any excipient which is nontoxic to the cell or mammal being exposed thereto at the dosages and concentrations employed. The pharmaceutical composition may include one or additional therapeutic agents.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

Pharmaceutically acceptable carriers include solvents, dispersion media, buffers, coatings, antibacterial and antifungal agents, wetting agents, preservatives, buggers, chelating agents, antioxidants, isotonic agents and absorption delaying agents.

Pharmaceutically acceptable carriers include water; saline; phosphate buffered saline; dextrose; glycerol; alcohols such as ethanol and isopropanol; phosphate, citrate and other organic acids; ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; EDTA; salt forming counterions such as sodium; and/or nonionic surfactants such as TWEEN, polyethylene glycol (PEG), and PLURONICS; isotonic agents such as sugars, polyalcohols such as mannitol and sorbitol, and sodium chloride; as well as combinations thereof.

Within the present invention, the disclosed compounds may be prepared in the form of pharmaceutically acceptable salts. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods well known in the art, e.g., by dissolving the free amine bases with an excess of the acid in aqueous alcohol, or neutralizing a free carboxylic acid with an alkali metal base such as a hydroxide, or with an amine.

Compounds described herein can be prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both amino and carboxy groups often exist in equilibrium with their zwitterionic forms. Thus, any of the compounds described herein that contain, for example, both amino and carboxy groups, also include reference to their corresponding zwitterions.

The pharmaceutical compositions of the invention may be formulated in a variety of ways, including for example, solid and semi-solid forms, such as tablets, pills, powders, gels, and gums. The composition is in a form suitable for oral administration. The composition may be formulated as an immediate, controlled, extended or delayed release composition.

In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

During the manufacturing, the carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

In some embodiments, the composition includes isotonic agents, for example, sugars, polyalcohols, such as mannitol, sorbitol, or sodium chloride. Prolonged absorption of the injectable compositions can be brought about by including in the composition an agent which delays absorption, for example, aluminum monostearate and gelatin.

Effective doses of the compositions of the present invention, for treatment of conditions or diseases as described herein vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

The pharmaceutical compositions of the invention may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

In one aspect, the dosage of tyrosine hydroxylase inhibitor may range from about 1 mg to about 4 g. In a particular embodiment, the dosage of tyrosine hydroxylase inhibitor may range from about 3 mg to about 1000 mg. In some suitable embodiments the drug is given in divided doses. In some suitable embodiments of the invention, 25 mg of the tyrosine hydroxylase inhibitor is administered. In one example, 60 mg of the tyrosine derivative can be administered orally. In another example, 0.25 mL of a 2 mg/mL suspension of the tyrosine derivative can be administered subcutaneously.

In alternate embodiments, the dosage of tyrosine hydroxylase inhibitor may range from 3-4 grams per day, as is well known in the art and fully described in, for example, U.S. Pat. No. 4,165,382 which is incorporated by reference herein in its entirety.

In some embodiments, the drug is administered divided in four doses. In other embodiments, the dosage of tyrosine hydroxylase inhibitor, in particular AMPT, may range from 1000 mg to 3000 mg per day, e.g., for treatment of pheochromocytoma, and a low dosage of AMPT of 1 gram per day or less (such as 250 mg/day to 325 mg/day, 250 mg b.i.d., or 250 mg three times per week in combination with other therapeutic agents, such as risperidone, haloperidol, clozapine or combinations thereof) for dystonia and dyskinesia, as is well known in the art and fully described in, for example, Demser (metyrosine) in PDR; Ankenman R. and M. F. Salvatore, J. Neuropsychiatry Clin. Neurosci, 2007; 19:65-69; and Oswald, J. N., et al., Challenge and Therapeutic Studies Using Alpha-methyl-para-Tyrosine (AMPT) in Neuropsychotic Disorders: A Review in Central Nervous System Agents in Medicinal Chemistry (formerly Current Medicinal Chemistry-Central Nervous System Agents), 8 (4), 249-256, 2008, which are incorporated by reference herein in their entirety.

In another aspect, the dosage of another agent useful in the treatment of a disease may include a therapeutically effective or clinically acceptable amount. In another example, the dosage of another agent is an amount that complements with or enhances the effect of a tyrosine hydroxylase inhibitor described herein.

As used herein, the terms “treat” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

In certain embodiments, the chewable and orally dissolvable and/or disintegrable product can be chewed by a user until substantially all of the ingredients contained therein substantially dissolve and disintegrate in the user's mouth.

The composition of the invention may be administered only once, or it may be administered multiple times. For multiple dosages, the composition may be, for example, administered three times a day, twice a day, once a day, once every two days, twice a week, weekly, once every two weeks, or monthly.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

“Administration” to a subject is not limited to any particular delivery system and may include, without limitation, oral administration (for example, in capsules, suspensions or tablets). Administration to a host may occur in a single dose or in repeat administrations, and in any of a variety of physiologically acceptable salt forms, and/or with an acceptable pharmaceutical carrier and/or additive as part of a pharmaceutical composition (described earlier). Once again, physiologically acceptable salt forms and standard pharmaceutical formulation techniques are well known to persons skilled in the art (see, for example, Remington's Pharmaceutical Sciences, Mack Publishing Co.).

### Manufacturing Methods

In various embodiments, the herein provided chewable compositions comprising a tyrosine hydroxylase inhibitor may be prepared by a granulation process in which powder particles of the active pharmaceutical ingredient (a tyrosine hydroxylase inhibitor) and of a chewability enhancing excipient(s) are made to adhere to each other, resulting in larger, multi-particle entities, called “granules”. The granules may have a particle size of between about 0.2 to about 4.0 mm. Granulation may be performed by either dry granulation or wet granulation. In dry granulation, a solid dosage form of a chewable pharmaceutical composition may be prepared by compressing (compacting by applying a force) a dry powder mixture of the tyrosine hydroxylase inhibitor and the chewability enhancing excipient, without the use of heat or solvent, to make enlarge the size of the compacts (the compressed powders). In some embodiments, dry granulation comprises slugging. In other embodiments, dry granulation comprises roller compaction, both of which processes are well known to those of ordinary skill in the art.

In alternate embodiments, the herein provided chewable compositions comprising a tyrosine hydroxylase inhibitor may be prepared by wet granulation, which comprises wet massing of the powder mixture of the tyrosine hydroxylase inhibitor and the chewability enhancing excipient(s) with a granulating liquid, wet sizing and drying. The granulating liquid may be water, ethanol, isopropyl alcohol or any other appropriate volatile solvent that is well known for use in wet granulation by those of ordinary skill in the art. The wet mass is pushed through a sieve to produce wet granules, which are dried.

In certain embodiments, the herein provided chewable compositions comprising a tyrosine hydroxylase inhibitor may be prepared by direct compression to produce a chewable tablet. In the direct compression process, the tyrosine hydroxylase inhibitor is mixed with the chewability enhancing excipient(s) and a lubricant(s), followed by compression.

In other embodiments, certain excipients may be co-processed, such as microcrystalline cellulose and a galacotomannan, which includes, but is not limited to guar gum, locust bean gum, cassia gum, tara gum, or a mixture thereof.

The formulations described herein can be used to treat any suitable mammal, including primates, such as monkeys and humans, horses, cows, cats, dogs, rabbits, and rodents such as rats and mice. In one embodiment, the mammal to be treated is human.

All patents and literature references cited in the present specification are hereby incorporated by reference in their entirety.

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### EXAMPLES

### Example 1

The following formulation method is an example of the preparation of a chewable α-methyl-DL-tyrosine tablet composition. The chewable formulation dissolves more quickly than, for example, a controlled-release formulation, such as an enteric coated formulation, or a conventional non-chewable tablet. The chewable formulation is prepared by a direct compression process, a dry granulation process, a wet granulation process, extrusion, or fluid-bed coating, followed by direct compression.

### Chewable Formulation of α-methyl-DL-tyrosine

### Chewable AMPT Tablet Formulation

| Ingredient | Mannitol-based formulation g/100 g (wt. %) |
|---|---|
| Alpha-methyl-para-tyrosine | 33.33 |
| Colloidal Silicon Dioxide, NF-M-5P | 0.85 |
| Sucralose, NF | 0.15 |
| Magnesium Stearate, NF | 1.35 |
| Crosscarmellose Sodium, NF Ac-DI-Sol SD-711 NF | 2.80 |
| Avicel CE-15 | 5.30 |
| Citric Acid, Anhydrous | 1.50 |
| Natural Orange Flavor #SC356177 | 0.45 |
| Mannitol, USP Pearlitol 100 SD | 54.27 |

In an embodiment, a total dose of AMPT of 6 g per day may be administered to a patient in which the AMPT dose may be divided into 3 administrations per day, for a dose of 2 gm AMPT per administration of a chewable AMPT tablet of 6 g (total tablet weight including the above-listed excipients adjusted accordingly, e.g., 3.256 g mannitol, e3tc.).

In an alternate embodiment, a 10 g chewable AMPT tablet is formulated comprising 3.33 g of AMPT and further comprising the above-listed ingredients in amounts reduced to one-tenth, e.g., 5.427 g mannitol is added to the 10 g formulation. The tablet components are further adjusted by a decrease of 10% w/w for a 3 g AMPT per 9 g chewable tablet (total tablet weight), thereby allowing for an administration of twice per day of the 9 g tablet.

### Example 2

The following formulation method is an example of the preparation of a soft chewable α-methyl-DL-tyrosine composition. The chewable formulation dissolves more quickly than, for example, a controlled-release formulation, such as an enteric coated formulation. The chewable formulation is prepared by a direct compression process, a dry granulation process, a wet granulation process, extrusion, or fluid-bed coating, followed by direct compression.

### Chewable Formulation of α-methyl-DL-tyrosine

### Soft Chewable AMPT Formulation

| Ingredient | Fructose-based formulation g/100 g (wt. %) |
|---|---|
| Alpha-methyl-para-tyrosine | 43.6 |
| Calcium Carbonate | 8.05 |
| Krystar ® Liquid Fructose | 26.6 |
| Tricalcium phosphate | 0 |
| Sucrose | 10.0 |
| Coloring Agent | 0 |
| Citric Acid, Anhydrous | 2.50 |
| Flavoring Agent | 0.04 |
| Glycerin | 2.18 |
| Soy Lecithin | 0.73 |
| Hydrogenated Coconut Oil | 6.0 |
| Mono- and Di-glycerides | 0.3 |

### Examples 3-5

### Chewable Formulations of α-Methyl-DL-Tyrosine

### Chewable AMPT Tablet Formulations

| Ingredient | Mannitol- and lactose-based formulation g/1000 g (wt. %) Example 3 (aqueous granulation) | Mannitol- and lactose-based formulation g/1000 g (wt. %) Example 4 (non-aqueous granulation) | Mannitol- based formulation g/1000 g (wt. %) Example 5 (Direct Compression) |
|---|---|---|---|
| Alpha-methyl-para- tyrosine | 500.0 | 500.0 | 500.0 |
| Mannitol | 100.0 | 150.0 | 150.0 |
| Lactose anhydrous | 100.0 | | |
| Avicel 101 (MCC) | 75.0 | 100.0 | 100.0 |
| Stevia | — | 20.0 | 20.0 |
| Aspartame | 15.0 | — | — |
| Magnesium stearate | 5.0 | 5.0 | 5.0 |
| PVP 10% | q.s.* | q.s.** | —*** |
| Talc | 5.0 | 5.0 | 5.0 |
| Citric Acid, Anhydrous | 3.0 | 3.0 | 3.0 |
| Vanillin/raspberry flavor | 5.0/0.0 | 5.0/0.0 | 5.0/5.0 |
| Raspberry color | — | — | 0.5 |

*q.s. = AMPT and excipients are blended for 2 min., and a sufficient amount of 10% PVP is added to make a dough mass. The dough is passed though sieve no. 12 to obtain raw granules, which are dried in a hot air oven at 50° C. for 30 min. After drying, the sieved granules are blended with aspartame, flavoring agent, coloring agent, magnesium stearate and talc. The granule mixture is evaluated for flow property and compressed using a single punch tableting machine (Cadmach CO. Pvt. Ltd.) equipped with 15 mm round flat and plain punch to achieve required hardness. No coloring agent is added.
**q.s. = AMPT and excipients are granulated with 10% PVP in isopropyl alcohol and dried a hot air oven at 40-50° C. for 20-30 min. The dried granules are passed through mesh no. 22 and blended with stevia; coloring agent and flavoring agent are added to the granules and blended for 10 min. The blend is lubricated with magnesium stearate and talc for 2 min. The powder blends are evaluated for flow properties and compressed into tablets. No coloring agent is added.
***The drug and Avicel, mannitol, starch and stevia are blended for 10 min.; flavoring agent and coloring agent are added to the mixture. The blend is then lubricated with magnesium stearate and talc for 2 min. The powder blends are evaluated for flow properties and compressed into tablets.

The hardness of the herein provided chewable tablet formulations is such that the tablets withstand the demands of manufacturing, packaging, shipping, and distribution, as well as being chewable without difficulty by the intended subject population (e.g., including the subject being a child or elderly, or a subject having difficulty swallowing). In an embodiment, the herein provided chewable tablet compositions have a hardness of <12 kp, as recommended by the FDA. (1 kilopond (kp)=1 kilogram-force (kgf)=9.8 Newton (N)=1.4 Strong-Cobb Units (scu)). The USP 39-NF34<1217> Tablet Breaking Force provides a public standard to ensure consistent measurement of the tablet hardness. In various embodiments, herein provided uncoated chewable tablet formulations may undergo friability testing (testing the durability of tablets during transit) and are inspected for chipping, crumbling or breakage and percentage of tablet mass lost through chipping. A maximum mean weight loss from three samples of not more than 1.0% and zero broken (such as chipped) tablets are deemed acceptable.

Having described preferred embodiments of the invention, it is to be understood that the invention is not limited to the precise embodiments, and that various changes and modifications may be effected therein by those skilled in the art without departing from the scope or spirit of the invention as defined in the appended claims.

What is claimed is:

1. A chewable formulation comprising a therapeutically effective amount of a tyrosine hydroxylase inhibitor and a chewability enhancing excipient.

2. The formulation of claim 1, wherein the chewability enhancing excipient comprises a disintegrant, a taste masking agent or a combination thereof.

3. The formulation of claim 2, wherein the disintegrant comprises a starch, a starch derivative; cellulose, sodium carboxymethyl cellulose (Na-CMC), a cellulose derivative, a crosslinked polymer, a clay, a cation exchange resin, fructose, povidone, a surfactant, a natural gum, or a combination thereof.

4. The formulation of claim 3, wherein natural gum is xanthan gum, alginate, chitosan, carrageenan, gellan gum, guar gum, gelatin, agar, alginate, carrageenan, gellan gum, gum Arabic, konjac gum, locust bean gum, modified starch, pectin or a combination thereof.

5. The formulation of claim 2, wherein the taste masking agent is a flavoring agent, a sweetener, a lipid, an acid or a combination thereof.

6. The formulation of claim 2, further comprising one or more of a binder, an adhesive, a diluent, a lubricant, an anti-adherent, a glidant, an adsorbent, a preservative, an antioxidant or a combination thereof.

7. The formulation of claim 1, further comprising a coloring agent.

8. The formulation of claim 1, further comprising an alkalinizing agent selected from the group consisting of sodium bicarbonate, ammonium chloride, calcium carbonate, sodium citrate/citric acid, potassium citrate/citric acid, and tricitrates comprising citric acid, potassium citrate and sodium citrate.

9. The formulation of claim 1, wherein said tyrosine hydroxylase inhibitor is racemic α-methyl-DL-tyrosine.

10. The formulation of claim 1, wherein said tyrosine hydroxylase inhibitor is metyrosine or α-methyl-L-tyrosine.

11. The formulation of claim 1, wherein said tyrosine hydroxylase inhibitor is α-methyl-D-tyrosine.

12. The formulation of claim 1, wherein said tyrosine hydroxylase inhibitor is a tyrosine derivative.

13. The formulation of claim 12, wherein said tyrosine derivative is methyl (2R)-2-amino-3-(2-chloro-4 hydroxyphenyl) propanoate, D-tyrosine ethyl ester hydrochloride, methyl (2R)-2-amino-3-(2,6-dichloro-3,4-dimethoxyphenyl) propanoate H-D-Tyr(TBU)-allyl ester HCl, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6-fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6-dichloro-3-hydroxy-4-methoxyphenyl) propanoate, methyl (2R)-2-amino-3-(3-chloro-4-hydroxyphenyl) propanoate, H-DL-tyr-OME HCl, H-3,5-diiodo-tyr-OME HCl, H-D-3,5-diiodo-tyr-OME HCl, H-D-tyr-OME HCl, D-tyrosine methyl ester hydrochloride, D-tyrosine-ome HCl, methyl D-tyrosinate hydrochloride, H-D-tyr-OMe HCl, D-tyrosine methyl ester HCl, H-D-Tyr-OMe-HCl, (2R)-2-amino-3-(4-hydroxyphenyl) propionic acid, (2R)-2-amino-3-(4-hydroxyphenyl) methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-Tyr (3,5-I2)-OSu, Fmoc-tyr(3-NO₂)—OH, α-methyl-L-tyrosine, α-methyl-D-tyrosine, α-methyl-DL-tyrosine, or a combination thereof.

14. The formulation of claim 1, wherein said tyrosine hydroxylase inhibitor is present in an amount of 150-300 mg.

15. The formulation of claim 1, wherein the tyrosine hydroxylase is administered in divided doses.

16. The formulation of claim 1, further comprising an effective amount of one or more another therapeutic agent.

17. The formulation of claim 16, wherein the one or more another therapeutic agent is an antidepressant, a benzodiazepine, a glucocorticoid, a cannabinoid or a combination thereof.

18. The formulation of claim 16, wherein at least one of said one or more another therapeutic agent is a vasopressin analog.

19. The formulation of claim 18, wherein the vasopressin analog is desompressin.

20. The formulation of claim 16, wherein the one or more another therapeutic agent is a neuromodulating agent.

21. The formulation of claim 20, wherein the neuromodulating agent is GABA.

22. The formulation of claim 20, wherein the neuromodulating agent potentiates acetylcholine.

23. The formulation of claim 20, wherein the neuromodulating agent is rivastigmine, or pilocarpine, or similar agents.

24. The formulation of claim 16, wherein said tyrosine hydroxylase inhibitor is racemic α-methyl-DL-tyrosine and wherein said one or more another therapeutic agent comprises desompressin and GABA.

25. The formulation of claim 17, wherein said antidepressant is a selective serotonin reuptake inhibitor (SSRI), a serotonin-norepinephrine reuptake inhibitor (SNRI), a tricyclic antidepressant, or a combination thereof.

26. The formulation of claim 17, wherein said antidepressant is sertraline, fluoxetine, paroxetine, venlafaxine, or a combination thereof.

27. A method for manufacturing the formulation of claim 1, the method comprising admixing the tyrosine hydroxylase inhibitor and the chewability enhancing excipient; and configuring the mixture into a unit dosage form.

28. The method of claim 27, wherein the chewability enhancing excipient is co-processed before the admixing.

29. The method of claim 27, wherein the mixture is configured by dry granulation or wet granulation.

30. The method of claim 27, wherein the mixture is configured by extrusion with compression or without compression.

31. The method of claim 27, further comprising admixing a lubricant with the tyrosine hydroxylase inhibitor and the chewability enhancing excipient.

32. The method of claim 27, wherein the mixture is configured by direct compression.

33. A method for treating a disease or disorder in a subject in need thereof, the method comprising administering to said subject the formulation of claim 1.
